(19) United States
(12) Patent Application Publication
(10) Pub. No.: US 2016/0202268 A1
(43) Pub. Date: Jul. 14, 2016

(54) METHOD OF DIAGNOSING KIDNEY DAMAGE IN DIABETIC PATIENTS USING URINARY CD36 AS A BIOMARKER

(71) Applicant: VIT University (Vellore Institute of Technology), Vellore, TN (US)

(72) Inventors: Shiju Thomas Michael, Nagercoil (IN); Pragasam Viswanathan, Vellore (IN)

(21) Appl. No.: 14/630,739

(22) Filed: Feb. 25, 2015

(30) Foreign Application Priority Data
Jan. 11, 2015 (IN) 166/CHE/2015

(51) Int. Cl. G01N 33/68; C07K 14/705

(52) U.S. Cl. CPC G01N 33/6872; C07K 14/70596; G01N 2333/70596; G01N 2800/56

(57) ABSTRACT

A method of diagnosing kidney damage in a subject using urinary CD36 as a biomarker is provided. The method includes following steps of: (a) providing a urine sample obtained from the subject who is suspected of having the kidney damage, (b) detecting a level of the urinary CD36 in the urine sample of the subject, and (c) determining whether the subject is suffering from the kidney damage based on the level of the urinary CD36 thus detected. An elevated level of the urinary CD36 in the subject relative to that in a healthy subject indicates that the subject is suffering from the kidney damage.

### BACKGROUND

1. Technical Field

The embodiments herein generally relate to a biomarker, and, more particularly, to a use of a urinary CD36 as a biomarker to diagnose kidney damage in a subject/patient.

2. Description of the Related Art

In humans, the kidneys are the main excretory organs that eliminate urea, citric acid, and other waste metabolites. The kidney participates in whole-body homeostasis, regulating acid-base balance, electrolyte concentrations, extracellular fluid volume, and blood pressure. The kidneys excrete a variety of waste products produced by metabolism into the urine. These include the nitrogenous wastes urea, from protein catabolism, and uric acid, from nucleic acid metabolism. The kidneys secrete a variety of hormones, including erythropoietin, and the enzyme renin.

Diabetic nephropathy is a progressive kidney disease caused by angiopathy of capillaries in the kidney glomeruli. The diabetic nephropathy is characterized by nephrotic syndrome and diffuse glomerulosclerosis. The diabetic nephropathy is due to longstanding diabetes mellitus, and is a prime reason for dialysis in many developed countries. The diabetic nephropathy is the leading cause of end stage kidney disease. About 20 million people have kidney failure as a result of diabetes around the world. The diabetic nephropathy is a severe late complication of both type1 diabetes (i.e. T1D) and type2 diabetes (i.e. T2D) which frequently requires kidney replacement therapy. The chronic kidney disease is estimated to affect 5-10% of adults in industrialized countries such as Unites States. Loss of kidney function results in retention of excess water and metabolic waste including urea in the human body. An albumin, a creatinine, a urea, a Neutrophil Gelatinase Associated Lipocalin (NGAL), a N-Acetyl Glucosaminidase (NAG), a Kidney Injury Molecule-1 (KIM-1), a cystatin-C, and a collagen IV are reported as urinary biomarkers of kidney damage. Even though, these urinary biomarkers are used to diagnose the kidney damage, it is less possible to diagnose at an earlier stage of the kidney damage. These urinary biomarkers are used to report the kidney damage only at the later stages. Accordingly, there remains a need for a biomarker that diagnoses kidney damage at the earlier stage in human beings/subjects/patients.

### SUMMARY

In view of a foregoing, an embodiment herein provides a method of diagnosing kidney damage in a subject using urinary CD36 as a biomarker. The method includes following steps of: (a) providing a urine sample obtained from the subject who is suspected of having said kidney damage, (b) detecting a level of the urinary CD36 in the urine sample of the subject, and (c) determining whether the subject is suffering from the kidney damage based on the level of the urinary CD36 thus detected. An elevated level of the urinary CD36 in the subject relative to a control value corresponding to a healthy subject indicates that the subject is suffering from the kidney damage.

In one embodiment, the level of the urinary CD36 in the urine sample of the subject is detected using Enzyme-Linked Immunosorbent Assay (ELISA) test.

In another embodiment, the subject is suffering from different stages of the kidney damage selected from at least one of (a) hypertrophy, (b) silent, (c) incipient, (d) microalbuminuria, and (e) macroalbuminuria. In yet another embodiment, the subject is a diabetic patient.

In yet another embodiment, the control value is selected from the group consisting of a value obtained from a healthy control individual, a panel of control values from a set of healthy individuals, and a stored set of data corresponding to control individuals that are not afflicted with the kidney damage.

In yet another embodiment, the method further includes the step of determining the level of the urinary CD36 in the urine sample of the subject to diagnose an earlier stage of the kidney damage in the subject.

In one aspect, an urinary CD36 biomarker for use in the diagnosis of kidney damage in a urine sample of a subject is provided. In one embodiment, a level of the urinary CD36 in the urine sample of the subject determines whether the subject is suffering from the kidney damage.

In another embodiment, an elevated level of the urinary CD36 in the subject relative to a control value corresponding to a healthy subject indicates that the subject is suffering from the kidney damage.

In yet another embodiment, the level of the urinary CD36 in the urine sample of the subject is detected using Enzyme-Linked Immunosorbent Assay (ELISA) test.

In yet another embodiment, the subject is suffering from different stages of the kidney damage selected from at least one of (a) hypertrophy, (b) silent, (c) incipient, (d) microalbuminuria, and (e) macroalbuminuria. In yet another embodiment, the subject is a diabetic patient.

In yet another embodiment, the control value is selected from the group consisting of a value obtained from a healthy control individual, a panel of control values from a set of healthy individuals, and a stored set of data corresponding to control individuals that are not afflicted with the kidney damage.

In another aspect, a method of diagnosing kidney damage in a subject using urinary CD36 as a biomarker is provided. The method includes following steps of: (a) providing a urine sample obtained from the subject who is suspected of having said kidney damage, (b) detecting a level of the urinary CD36 in the urine sample of the subject using Enzyme-Linked Immunosorbent Assay (ELISA) test, and (c) determining whether the subject is suffering from the kidney damage based on the level of the urinary CD36 thus detected. An elevated level of the urinary CD36 in the subject relative to a control value corresponding to a healthy subject indicates that the subject is suffering from the kidney damage.

In one embodiment, the method further includes the step of determining the level of the urinary CD36 in the urine sample of the subject to diagnose an earlier stage of the kidney damage in the subject.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a graphical representation that illustrates a level of urinary CD36 in a urine sample of a subject/patient suffering from kidney damage at different stages according to an embodiment herein;

FIG. 2A through 2C illustrate graphical representations that illustrates a correlation of the level of the urinary CD36 with other kidney damage biomarkers such as an albumin, a creatinine, and a urea according to an embodiment herein;

FIG. 3 is a graphical representation that illustrates a level of urinary CD36 in a urine sample of a rat suffering from kidney damage at different stages according to an embodiment herein; and

FIG. 4 is a graphical representation that illustrates the level of the CD36 in the kidney of the rat according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

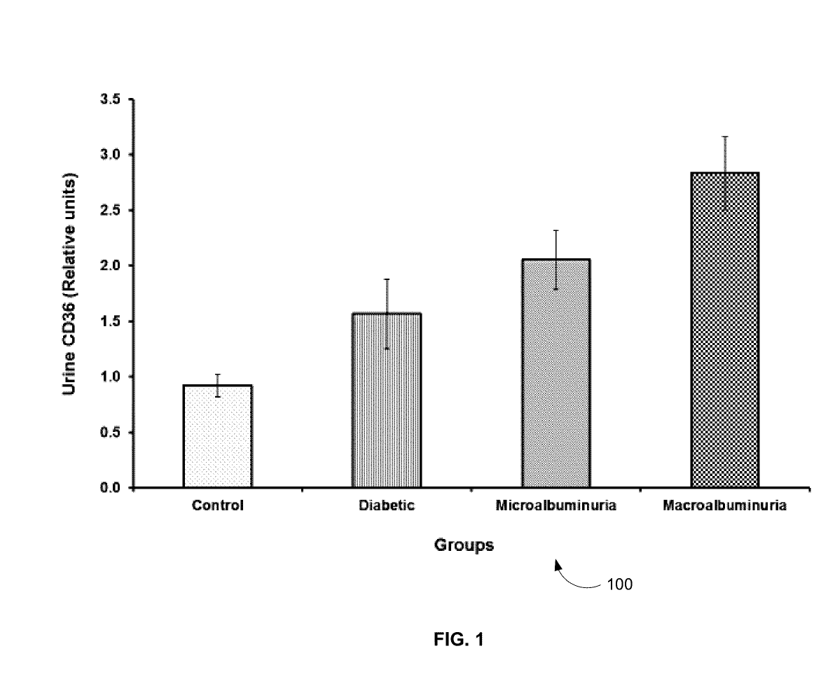

As mentioned, there remains a need for a biomarker that diagnoses the kidney damage at the earlier stage in subjects/patients, and assessing efficacy of kidney damage treatment. The embodiments herein achieve this by using urinary CD36 as a biomarker for diagnosing the kidney damage in the subject. The urinary CD36 as a biomarker diagnoses the kidney damage in the subject at an earlier stage. Further, the different stages of the kidney damage are diagnosed based on a level of the urinary CD36 in the urine sample of the subject/patient. Referring now to the drawings, and more particularly to FIGS. 1 through 4, where similar reference characters denote corresponding features consistently throughout the figures, there are shown preferred embodiments.

### DEFINITIONS

Diagnosis is an estimation and/or determination of whether or not a patient is suffering from a given disease or condition, such as kidney damage or diabetic nephropathy. The skilled human being makes the diagnosis on the basis of one or more diagnostic indicators, i.e., in the amount or concentration of a biomarker, or change in amount of biomarker which is indicative of the presence, severity, or absence of the condition.

The term “correlating” as used herein, refers to comparing the presence or amount of the biomarker(s) in the patient to its presence or amount in individuals suffering from or at risk of suffering from, a given condition; or in persons known to be free of a given condition. As discussed above, a biomarker level in the patient sample can be compared to a level known to be associated with a specific diagnosis. The sample's biomarker level is said to have been correlated with a diagnosis; that is, the skilled person can use the biomarker level to determine whether the patient suffers from a specific type of the kidney damage, and respond accordingly. Alternatively, the sample's biomarker level can be compared to a biomarker level known to be associated with a good outcome (e.g., the absence of disease, etc.).

It is appreciated that the term ‘biomarker’ in the present embodiment refers to the urinary CD36. The biomarker is an indicator of normal biological processes, pathogenic processes or pharmacological responses to a therapeutic intervention without necessarily being causally related to the clinical endpoint. The biomarker is used as diagnostic tools, staging tools, and prognostic tools. The embodiment herein uses the urinary CD36 as a biomarker for diagnosing the kidney damage, and assessing efficacy of the kidney damage treatment, and for manufacturing kits used for the just-mentioned purposes. The embodiment herein is based on the unexpected discoveries that the urinary CD36 levels are elevated in the urine sample of the patients suffering from various kidney damages as compared to healthy subjects.

The term control or control expression level refers to a baseline level of the urinary CD36 as a biomarker as determined from one i.e. healthy or normal subjects believed not to or confirmed by diagnosis not to have the kidney damage. For example, the level of the urinary CD36 in the urine sample collected from diabetic patients is high when compared to the urinary CD36 level in the urine sample that is obtained from the healthy or normal subjects.

The term “determining the diagnosis” as used herein refers to methods by which the skilled person can determine the presence or absence of a particular disease or condition, such as the kidney damage, in the patient. The term “diagnosis” does not refer to the ability to determine the presence or absence of a particular condition or disease with 100% accuracy. Instead, the skilled person will understand that the term “diagnosis” refers to an increased probability that a certain disease or condition is present in the subject. In preferred embodiments, a diagnosis indicates about a 5% increased chance that a disease or condition is present, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−2%.

The embodiment herein provides a method of diagnosing different stages of the kidney damage (e.g., hypertrophy, silent, incipient, microalbuminuria and macroalbuminuria) in a subject/patient using urinary CD36 as a biomarker. This method includes (i) providing a urine sample obtained from a subject/patient, (ii) detecting the level of the urinary CD36 in the urine sample, and (iii) determining whether the subject/patient is suffering from the kidney damage based on the level of the urinary CD36 level thus detected. An elevated level of the urinary CD36 in the subject/patient relative to that in a healthy subject indicates that the subject is suffering from the kidney damage. The level of the urinary CD36 in the urine sample of the subject/patient is determined using ELISA test.

FIG. 1 is a graphical representation 100 that illustrates a level of urinary CD36 in a urine sample of a subject/patient suffering from kidney damage at different stages according to an embodiment herein. The different stages of the kidney damage in the subject are plotted in X-axis. The level of the urinary CD36 in the urine sample of the subject/patient is plotted in Y-axis. The level of the urinary CD36 in the urine sample is average in the normal healthy subject. The level of the urinary CD36 in the urine sample is high when the subject is suffering from diabetes. In other words, the level of the urinary CD36 in the urine sample of the subject who is suffering from the diabetes is high when compared to the level of the urinary CD36 in urine sample of the normal healthy subject. The level of the urinary CD36 in the urine sample of the subject who is suffering from microalbuminuria is high when compared to the level of the urinary CD36 in the urine sample of the subject who is suffering from the diabetes. The level of the urinary CD36 in the urine sample of the subject who is suffering from macroalbuminuria is high when compared to the level of the urinary CD36 in the urine sample of the subject who is suffering from microalbuminuria, as in the FIG. 1. A level of the significance of the urinary CD36 in the urine sample of the subject who is suffering from the microalbuminuria is 0.01 (i.e. P<0.01). The level of the significance of the urinary CD36 in the urine sample of the subject who is suffering from the macroalbuminuria is 0.001 (i.e. P<0.001) when compared to the normal healthy subject.

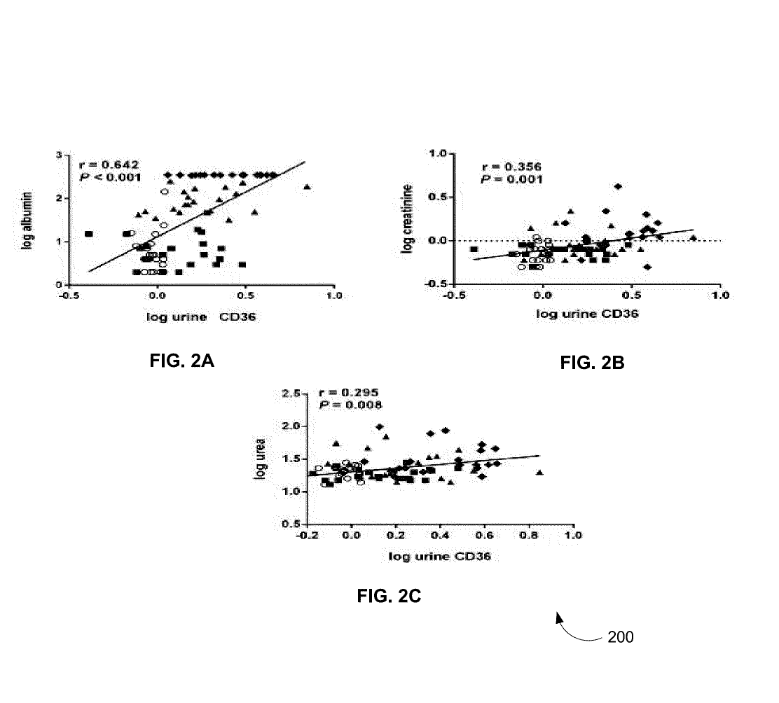

FIG. 2A through 2C illustrate graphical representations 200 that illustrates a correlation of the level of the urinary CD36 with other kidney damage biomarkers such as an albumin, a creatinine, and a urea according to an embodiment herein. The level of the urinary CD36 in the urine sample of the subject at the different stages of the kidney damage is showed in positive correlation when compared to the other standard kidney damage biomarkers such as the albumin, the creatinine, and the urea. In the FIG. 2A, the level of the urinary CD36 in the urine sample of the subject is plotted in X-axis and the level of the albumin in the urine sample of the subject is plotted in Y-axis. From FIG. 2A, the results indicate that the level of the urinary CD36 in the urine sample of the subject is similar to the level of the albumin in the urine sample of the subject.

In the FIG. 2B, the level of the urinary CD36 in the urine sample of the subject is plotted in X-axis and the level of the creatinine in the urine sample of the subject is plotted in Y-axis. From FIG. 2B, the results indicate that the level of the urinary CD36 in the urine sample of the subject is similar to the level of the creatinine in the urine sample of the subject. In the FIG. 2C, the level of the urinary CD36 in the urine sample of the subject is plotted in X-axis, and the level of the urea in the urine sample of the subject is plotted in Y-axis. From FIG. 2C, the results indicate that the level of the urinary CD36 in the urine sample of the subject is similar to the level of the urea in the urine sample of the subject. The Pearson correlation coefficient (r) and the level of the significance (P) for the correlation of the level of the urinary CD36 with the level of the albumin is 0.642, and 0.000 respectively (i.e. r=0.642, P=0.000). Similarly, the Pearson correlation coefficient (r) and the level of the significance (P) for the correlation of the level of the urinary CD36 with the level of the creatinine is 0.356, and 0.001 respectively (i.e. r=0.356, P=0.001). Similarly, the Pearson correlation coefficient (r) and the level of the significance (P) for the correlation of the level of the urinary CD36 with the level of the urea is 0.295, and 0.008 respectively (i.e. r=0.295, P=0.008).

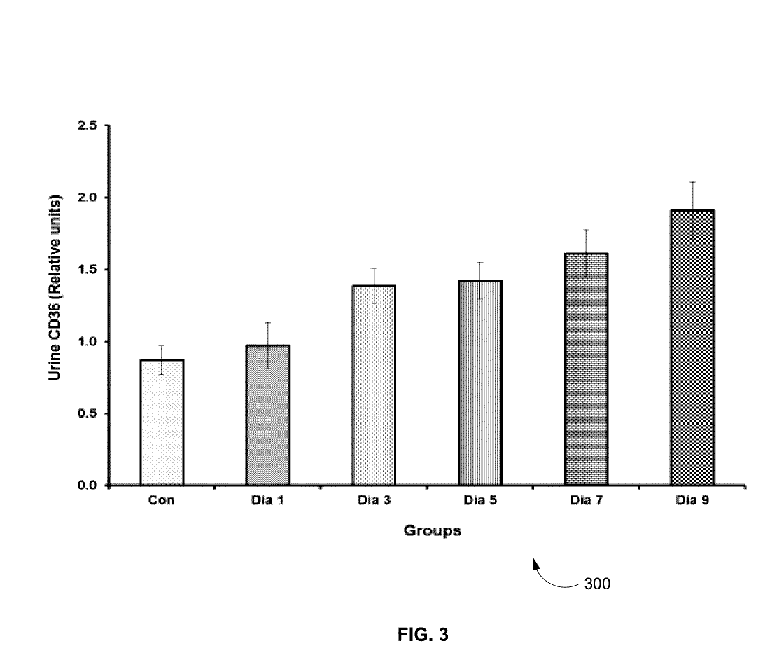

FIG. 3 is a graphical representation 300 that illustrates a level of urinary CD36 in a urine sample of a rat suffering from kidney damage at different stages according to an embodiment herein. The different stages of the kidney damage in the rat are plotted in X-axis. The level of the urinary CD36 in the urine sample of the rat is plotted in Y-axis. The level of the urinary CD36 in the urine sample is average in the healthy rat. The level of the urinary CD36 in the urine sample is high when the rat is suffering from diabetes at first month (i.e. Dial). In other words, the level of the urinary CD36 in the urine sample of the rat which is suffering from diabetes at the first month is high when compared to the level of the urinary CD36 in urine sample of the normal healthy rat. The level of the urinary CD36 in the urine sample of the rat which is suffering from diabetes at third month (i.e. Dia3) is high when compared to the level of the urinary CD36 in the urine sample of the rat which is suffering from diabetes at the first month. The level of the urinary CD36 in the urine sample of the rat which is suffering from diabetes at fifth month (i.e. Dia5) is high when compared to the level of the urinary CD36 in the urine sample of the rat which is suffering from diabetes at third month.

The level of the urinary CD36 in the urine sample of the rat which is suffering from diabetes at seventh month (i.e. Dia7) is high when compared to the level of the urinary CD36 in the urine sample of the rat which is suffering from diabetes at the fifth month. The level of the urinary CD36 in the urine sample of the rat which is suffering from diabetes at ninth month (i.e. Dia9) is high when compared to the level of the urinary CD36 in the urine sample of the rat which is suffering from diabetes at the seventh month. From FIG. 3, the results indicate that the level of the urinary CD36 in the urine sample of the rat are elevated/increased with respect to increase in number of months that the rat is suffering with the diabetes. The level of significance of the urinary CD36 in the urine sample of the rat which is suffering from diabetes at the first month is 0.05 (i.e. P<0.05). The level of the significance of the urinary CD36 in the urine sample of the rat which is suffering from diabetes at the end of the third month is 0.01 (i.e. P<0.01). The level of the significance of the urinary CD36 in the urine sample of the rat which is suffering from diabetes at the end of the ninth month is 0.001 (i.e. P<0.001). From FIG. 3, the results indicate that the level of the significance of the urinary CD36 in the urine sample of the rat at different stages of the kidney damage is similar to the level of the significance of the urinary CD36 in the urine sample of the human at different stages of the kidney damage.

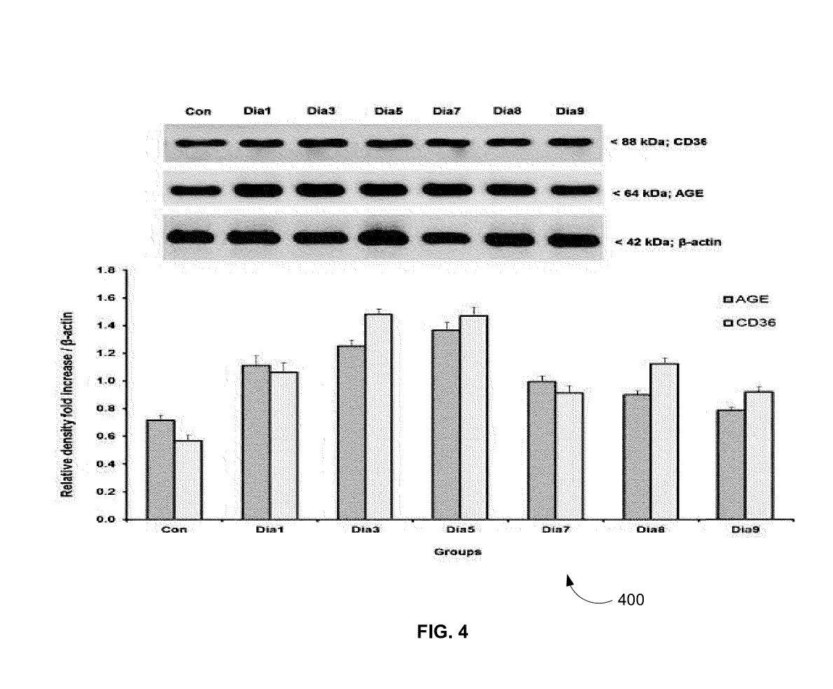

FIG. 4 is a graphical representation 400 that illustrates the level of the CD36 in the kidney of the rat according to an embodiment herein. The different stages of the kidney damage in the rat are plotted in X-axis. A relative increase in the density of the level of AGE (i.e. Advanced Glycation End products) and the level of the CD36 with respect to a beta-actin is plotted in Y-axis. The density of the level of the CD36 and the AGE observed in a western blotting is compared with the density of the level of the beta-actin. In one embodiment, the beta-actin is used as a house-keeping protein. The level of the beta-actin remains unaltered with respect to the different stages of the kidney damage as shown in the FIG. 4. During the initial stages of the kidney damage in the rat such as diabetes at first month (Dial), diabetes at third month (Dia3), and diabetes at fifth month (Dia5), the level of the AGE and the CD36 is increased in the kidney of the rat. During the final stages of the kidney damage in the rat such as diabetes at seventh month (Dia7), diabetes at eighth month (Dia8), and diabetes at ninth month (Dia9)), the level of the AGE and the CD36 is decreased in the kidney of the rat. The level of the AGE and the CD36 is decreased because of loss of proteins which is attributed to the kidney damage in the rat. The kidney damage in the diabetic rat in-turn influenced the increased level of the urinary CD36 in the urine sample of the rat.

The kidney damage in the subject/patient is diagnosed using a level of the urinary CD36 in the urine sample as a biomarker. The kidney damage of the subject/patient is diagnosed at the initial stage by identifying the level of the urinary CD36 in the urine sample of the subject. The use of the urinary CD36 as a biomarker to diagnose the kidney damage is easy, and rapid.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A method of diagnosing kidney damage in a subject using urinary CD36 as a biomarker, comprising:
providing a urine sample obtained from said subject, wherein said subject is suspected of having said kidney damage;
detecting a level of said urinary CD36 in said urine sample of said subject; and
determining whether said subject is suffering from said kidney damage based on said level of said urinary CD36 thus detected, wherein an elevated level of said urinary CD36 in said subject relative to a control value corresponding to a healthy subject indicates that said subject is suffering from said kidney damage.

2. The method of claim 1, wherein said level of said urinary CD36 in said urine sample of said subject is detected using Enzyme-Linked Immunosorbent Assay (ELISA) test.

3. The method of claim 1, wherein said subject is suffering from different stages of said kidney damage selected from at least one of (a) hypertrophy, (b) silent, (c) incipient, (d) microalbuminuria, and (e) macroalbuminuria.

4. The method of claim 1, wherein said subject is a diabetic patient.

5. The method of claim 1, wherein said control value is selected from the group consisting of a value obtained from a healthy control individual, a panel of control values from a set of healthy individuals, and a stored set of data corresponding to control individuals that are not afflicted with said kidney damage.

6. The method of claim 1, further comprising the step of determining said level of said urinary CD36 in said urine sample of said subject to diagnose an earlier stage of said kidney damage in said subject.

7. An urinary CD36 biomarker for use in the diagnosis of kidney damage in a urine sample of a subject.

8. An urinary CD36 biomarker for use according to claim 7, wherein a level of said urinary CD36 in said urine sample of said subject determines whether said subject is suffering from said kidney damage.

9. An urinary CD36 biomarker for use according to claim 8, wherein an elevated level of said urinary CD36 in said subject relative to a control value corresponding to a healthy subject indicates that said subject is suffering from said kidney damage.

10. An urinary CD36 biomarker for use according to claim 8, wherein said level of said urinary CD36 in said urine sample of said subject is detected using Enzyme-Linked Immunosorbent Assay (ELISA) test.

11. An urinary CD36 biomarker for use according to claim 7, wherein said subject is suffering from different stages of said kidney damage selected from at least one of (a) hypertrophy, (b) silent, (c) incipient, (d) microalbuminuria, and (e) macroalbuminuria.

12. An urinary CD36 biomarker for use according to claim 7, wherein said subject is a diabetic patient.

13. An urinary CD36 biomarker for use according to claim 9, wherein said control value is selected from the group consisting of a value obtained from a healthy control individual, a panel of control values from a set of healthy individuals, and a stored set of data corresponding to control individuals that are not afflicted with said kidney damage.

14. A method of diagnosing kidney damage in a subject using urinary CD36 as a biomarker, comprising:
providing a urine sample obtained from said subject, wherein said subject is suspected of having said kidney damage;
detecting a level of said urinary CD36 in said urine sample of said subject using Enzyme-Linked Immunosorbent Assay (ELISA) test; and
determining whether said subject is suffering from said kidney damage based on said level of said urinary CD36 thus detected, wherein an elevated level of said urinary CD36 in said subject relative to a control value corresponding to a healthy subject indicates that said subject is suffering from said kidney damage.

15. The method of claim 14, further comprising the step of determining said level of said urinary CD36 in said urine sample of said subject to diagnose an earlier stage of said kidney damage in said subject.
